(19) United States
(12) Patent Application Publication
(10) Pub. No.: US 2019/0209327 A1
(43) Pub. Date: Jul. 11, 2019

(54) AN IMPLANT FOR REPAIR OF BONE DEFECTS

(71) Applicant: FITZBIONICS LIMITED, Surrey (GB)

(72) Inventors: Noel Fitzpatrick, Surrey (GB); Jayantilal Meswania, Surrey (GB); Gordon Blunn, Surrey (GB)

(73) Assignee: FITZBIONICS LIMITED, Surrrey (GB)

(21) Appl. No.: 16/326,246

(22) PCT Filed: Aug. 16, 2017
(86) PCT No.: PCT/GB2017/052408

§ 371 Date: Feb. 18, 2019

(30) Foreign Application Priority Data
Aug. 18, 2016 (GB) 1614171.5

(51) Int. Cl. A61F 2/28; A61L 27/56

(52) U.S. Cl. CPC A61F 2/2846; A61L 27/56; A61F 2002/30011; A61L 2300/64; A61F 2002/2835

(57) ABSTRACT

An implant for repairing a segmental defect in a subject's bone at a segmental defect site, the implant comprising a scaffold for implantation at the segmental defect site and the implant optionally comprising: a plate for internal fixation, the plate being attachable to or integral with the scaffold, and the plate having means for securing it to bone outside of the segmental defect site; a hollow cavity within the scaffold; a plurality of longitudinal channels within the scaffold; and or a membrane received around the scaffold.

### CROSS REFERENCE TO RELATED APPLICATIONS

The present application is the U.S. national stage application of International Application PCT/GB2017/052408, filed Aug. 16, 2017, which international application was published on Feb. 22, 2018 as International Publication WO 2018/033725 A1. The International Application claims priority to Great Britain Application 1614171.5, filed Aug. 18, 2016.

### FIELD OF THE INVENTION

The invention relates to an implant for repairing a segmental defect in a subject's bone at a segmental defect site. The invention also relates to membranes for such implants and methods for implanting such implants.

### BACKGROUND TO THE INVENTION

Bone defects may result from a variety of causes. They may be due to trauma, bone infection, congenital defects or extensive excision of malignant tumours. Management of bone defects is very challenging. Small size defects can be easily managed by non-vascularized cancellous bone-grafting. The critical size for non-vascularized bone-grafting is 6.0-7.0 cm. Larger defects require other options.

Surgical options available for managing large defects are; vascularized bone-grafts, bone transport, non-vascularized grafts, allografts and fibular pro-tibia grafting. Vascularized bone-grafting is technically demanding and requires micro vascular surgical skills. The technique is reliable but the donor sites are limited. The advantage is that the bone can be transferred together with soft tissue to cover local soft tissue defect. Bone transport is a well-established technique for the management of very large bone defects. The procedure has a very high complication rate: up to 80%. Allografts complication rate is also high; may reach 50%.

The Masquelet technique is a relative new technique used in the management of large bone defects. It is based on two principles or operative stages:

1. The formation of induction membrane. After bone debridement, the defect is filled with bone cement. The cement is kept for a period of eight weeks. This allows the formation of induction membrane.

2. Cancellous bone grafting. After a period of eight weeks, the bone cement is gently removed. The defect is filled with cancellous bone graft. Defects as large as 25 cm can be managed using the Masquelet technique.

The first step in this technique is the formation of induction membrane. It forms around the bone cement. The membrane serves a very critical function: protection of cancellous bone graft from the body's immune system. This prevents cancellous bone resorption. This induction membrane has other features that may be important for bone union. Experimental work has indicated that the induction membrane is not inert. It is a living tissue that plays an important role in bone healing or union. However the induction membrane does not have the properties of a true periosteum.

The first stage in Masquelet's technique is mechanical: the bone cement provides additional support to the limb and maintains the defect. An intramedullary device may also be used as a primary stabilizer. The induction membrane is formed at this stage around the cement. The second stage is a biological one. This is the stage that has been studied extensively in a large number of experiments. It has been demonstrated that periosteal flap wrapped around the cancellous bone exerts a protective effect against bone resorption in muscle tissue. The cancellous bone is capable of forming bone even without stress to the bone. But the cancellous bone will resorb if the recipient bed is poorly vascularized.

The concept of creating a secluded anatomic site with the aim to promote healing was first introduced 50 years ago, when cellulose acetate filters were experimentally used for the regeneration of nerves and tendons. Murray et al. (1957) reported new bone formation beneath plastic cages adapted over decorticated femoral defects in the dog (Murray, G., Holden, R. & Roshlau, W. (1957) Experimental and clinical study of new growth of bone in a cavity. American Journal of Surgery 93: 385-387). Experimentally an occlusive PTFE membrane has been used to prevent wear particles from accessing the bone implant interface and for augmenting bone formation around implants (Bhumbra R P, Walker P S, Berman A B, Emmanual J, Barrett D S, Blunn G W. Prevention of loosening in total hip replacements using guided bone regeneration. Clin Orthop Relat Res. 2000 March; (372):192-204 and Bhumbra R S, Berman A B, Walker P S, Barrett D S, Blunn G W. Enhanced bone regeneration and formation around implants using guided bone regeneration. J Biomed Mater Res. 1998; 43(2):162-7).

An option for repairing bone defects that reduces the complexity of the Masquelet procedure would be desirable.

The term subject as referred to herein may refer to a human or animal subject.

### SUMMARY OF INVENTION

According to a first aspect of the invention there is provided an implant for repairing a segmental defect in a subject's bone at a segmental defect site, the implant comprising: a scaffold for implantation at the segmental defect site, the scaffold comprising a body, the body having a first end and a second end, the first and second ends being first and second open ends respectively, the scaffold further comprising a cavity extending between said first and second open ends.

The implant is suitably manufactured with a void between its two open ends, which allows the invasion of bone marrow into the cavity via the first and second open ends when implanted. Allograft or autograft material may optionally be packed into the cavity before it is implanted into the subject's bone, although in preferred embodiments the scaffold is implanted in the subject with the scaffold cavity empty. Bone marrow will invade into the cavity via the first and second open ends of the scaffold when implanted. The cavity is preferably a central cavity communicating with the first and second open ends. The scaffold suitably has an inner diameter defining the cavity and an outer diameter, the ratio of the inner diameter to the outer diameter preferably being around 1:1.5 to 1:5, more preferably around 1:3 to 1:5.

Preferably said first and second ends of the scaffold are opposing ends. The scaffold suitably has a longitudinal axis between the first and second ends of the scaffold. The longitudinal axis may be substantially straight or it may be curved, to substantially match the geometry of the bone into which the scaffold is to be implanted.

Preferably each of said first and second open ends of the scaffold is configured to engage bone at the segmental defect site when implanted.

Preferably each of said first and second open ends of the scaffold is configured to abut bone at the segmental defect site when implanted. In other words, the first end of the scaffold borders a first bone fragment on one side of the defect site and the second end of the scaffold borders a second bone fragment on the other side of the defect site when implanted. When implanted the first end of the scaffold is adjacent the distal end of the defect site and the second end of the scaffold is adjacent the proximal end of the defect site.

Preferably the length of the scaffold between its first and second ends is selected such that the first end of the scaffold abuts bone at one side of the segmental defect site and the second end of the scaffold abuts bone at the other side of the segmental defect site when implanted. Suitably the first end of the scaffold abuts a first bone fragment on one side of the defect site and the second end of the scaffold abuts a second bone fragment on the other side of the defect site when implanted.

Preferably the implant is configured for implantation at a segmental defect site in a subject's long bone. In preferred embodiments, the implant is suitably configured for implanting between a first bone fragment of a long bone distal of the defect site and a second bone fragment of said same long bone, proximal of the defect site, the first and second bone fragments being spaced apart from one another by the defect site.

Suitably the implant is configured for implantation at a segmental defect site in a subject's long bone, the long bone having a first bone fragment distal of the defect site and a second bone fragment proximal of the defect site, the scaffold being sized to be implanted between the first and second bone fragments. The length of the scaffold between its first and second ends is preferably selected to correspond to the spacing between the first and second bone fragments. The first bone fragment suitably has a first bone fragment face facing towards the defect site and the second bone fragment has a second bone fragment face facing towards the defect site, the length of the scaffold between its first and second ends preferably being selected such that the first end of the scaffold butts up against the first bone fragment face and the second end of the scaffold butts up against the second bone fragment face.

The implant may be custom manufactured to match the length of a particular segmental defect site of a subject or alternatively a set of implants comprising scaffolds of differing lengths can be provided from which an implant may be selected having a length that substantially matches the defect site. The defect site may not require preparation or alternatively it may be prepared before implantation to remove diseased or damaged bone. Where the bone is prepared before implantation, the length of the defect site between the first and second bone fragments is the length between the prepared ends of the bone.

Preferably the cavity in the scaffold has a longitudinal axis, the implant being configured such that the longitudinal axis of the cavity substantially aligns with a longitudinal axis of the subject's long bone at the defect site when implanted in said long bone.

Preferably the cavity is configured to align with the medullary cavity of said long bone when implanted. In other words, the implant is configured such that when implanted at a segmental defect site in a subject's long bone, the longitudinal axis of the cavity is substantially collinear with the longitudinal axis of the subject's long bone. Suitably, the longitudinal axis of the cavity is configured to substantially match the anatomical path of a natural bone marrow cavity between the first and second bone fragments if there were no defect in the bone. The longitudinal axis of the cavity may be curved or substantially straight depending on the longitudinal axis of the medullary cavity of the bone into which the implant is to be implanted.

Preferably the cross-sectional size and/or shape of the cavity is selected to substantially correspond with the cross-sectional size and/or shape of the medullary cavity of said long bone when implanted. Suitably the cross-sectional diameter of the cavity is selected to substantially correspond with the cross-sectional diameter of the subject's long bone medullary cavity in which the implant is to be implanted.

Preferably the cross-sectional size and/or shape of the scaffold is selected to substantially correspond with the cross-sectional size and/or shape of said subject's long bone in which it is to be implanted. In other words, the cross-sectional diameter of the scaffold is selected to substantially correspond with the cross-sectional diameter of said long bone. The implant may be custom manufactured such that the diameter and/or shape of the cavity substantially matches the diameter and/or shape of the medullary cavity in the long bone at said segmental defect site and/or the diameter and/or shape of the scaffold body substantially matches the cross-sectional diameter and/or shape of the long bone at the defect site. Alternatively a set of implants comprising scaffolds having cavities of differing diameters and/or having differing scaffold body diameters can be provided from which an implant may be selected having a cavity diameter that substantially matches the diameter of the medullary cavity at the defect site and/or a body diameter that substantially matches the diameter of the bone at the defect site.

Preferably the scaffold has a substantially circular cross-sectional shape transverse to a longitudinal axis between its first and second ends. Alternatively the scaffold may have a cross-sectional shape that substantially matches the anatomical cross-section of the bone into which it is to be implanted.

Preferably the scaffold is substantially rigid. Suitably the scaffold is sufficiently rigid such that it does not deform when forces that are likely to be imparted to the bone at the defect site during normal use are imparted to the scaffold, whilst being sufficiently flexible to allow physiological strains in the new forming bone. In other embodiments the stiffness of the scaffold may be augmented with extra-cortical plates which may be removed once bone has grown into the scaffold.

Preferably said cavity of the scaffold is configured to be empty when implanted in a subject. Suitably the cavity is free from allograft or autograft material when implanted into the subject's bone.

Preferably the implant further comprises a plate for internal fixation, the plate being attachable to or integral with the scaffold, and the plate having means for securing it to bone outside of the segmental defect site.

In some embodiments the plate and scaffold are integrally formed. Preferably the plate has a bone facing side and an opposing side, the scaffold projecting from the bone facing side of the plate. The plate extends from the scaffold in at least one direction. The plate may have a scaffold portion which overlies the scaffold and at least a first extension portion which extends from the scaffold to overlie bone when implanted. Preferably the implant is configured such that the bone facing side of the plate closely faces the bone or contacts bone outside of the segmental defect site.

In some embodiments the plate is attachable to the scaffold. In this case the implant suitably includes means for attaching the plate to the scaffold. The means for attaching may be through holes for receiving screws for attaching the plate to the scaffold, or some other means for attaching, such as a tie or strap for coupling the scaffold to the plate. Where a tie or strap is used, the tie or strap is received around the scaffold and plate and preferably fastened to attach them together. Preferably plate has a scaffold portion which overlies the scaffold when implanted, the scaffold portion of the plate having at least one through hole for receiving a screw for attaching the plate to the scaffold. Preferably the plate is attached to the scaffold in use using first and second screws, received in first and second through holes in the scaffold portion of the plate. Preferably the plate has a bone facing side and an opposing side, the implant being configured such that the bone facing side may be spaced away from the scaffold when implanted.

In the embodiments described above, the plate may be configured to receive at least one fixation element for securing the plate to bone. Alternatively the plate may include at least one integral fixation element. The plate may have at least one through hole for receiving a screw for securing the plate to bone. The plate may have a scaffold portion which the scaffold projects from, a first extension portion which extends from the scaffold in a first direction and a second extension portion which extends from the scaffold in a substantially opposite direction from the first extension portion, the plate having at least one through hole in the first extension portion and at least one through hole in the second extension portion. In this way the plate can be fixed to bone on either side of the segmental defect site. The or each at least one through hole for receiving a screw for securing the plate to bone may be threaded for engagement with a screw having a head that is externally threaded. Such screws with a head that is externally threaded for engagement with an internally threaded screw hole are called locking screws. The or each through hole in the plate for receiving a screw for attaching the plate to the scaffold may also be threaded screw holes for receiving locking screws.

According to a further aspect of the invention there is provided an implant for repairing a segmental defect in a subject's bone at a segmental defect site, the implant comprising: a scaffold for implantation at the segmental defect site, the scaffold comprising a body, the body having a first end, a second end and a longitudinal axis between the first and second ends, the scaffold further comprising a plurality of channels within its body, the plurality of channels being substantially aligned with a longitudinal axis of the scaffold.

The channels define longitudinal voids in the scaffold into which bone may grow when implanted. The channels may be cylindrical or may have cross-sectional shapes other than circular. For example, the channels may have square, triangular or complex cross-sectional shapes. The channels may be through pores that have first and second open ends or may be blind channels. The implant may be configured for implantation at a segmental defect site in a subject's long bone, the implant being configured such that the longitudinal axis of the scaffold substantially aligns with a longitudinal axis of the subject's long bone when implanted in said long bone. The longitudinal axis of the scaffold may be substantially straight or curved, to substantially match the geometry of the bone into which the scaffold is to be implanted.

The implant with channels in the scaffold aligned with the longitudinal axis of the scaffold may optionally have a plate for internal fixation and/or a hollow longitudinal cavity (larger in diameter than the channels) as described above.

Preferably each of the plurality of channels has a diameter of up to around 1000 micrometres, and more preferably around 150 to 400 micrometres. Preferably the diameter of each channel is around 200 to 350 micrometres. The scaffold is preferably porous both longitudinally and transversely, the longitudinal channels helping bone to grow and the transverse porosity allowing blood vessel ingrowth. The scaffold is preferably more porous in a transverse plane than in a longitudinal plane. For example, the scaffold may preferably have a porosity of up to around 60% in the transverse plane and up to around 30% in the longitudinal plane.

Preferably the scaffold is porous and the plurality of channels are formed by interconnected pores in the porous structure.

In preferred embodiments the scaffold body is made of a three dimensional mesh or lattice structure comprising struts intersecting at nodes, providing pores between the struts, the pores being microscale or nanoscale pores. The pores in the scaffold body may be of differing sizes, but generally having a dimension that is preferably up to around 1.5 mm in diameter. In preferred embodiments the pores will be between around 0.3 to 1.5 mm in size, more preferably between around 0.4 to 0.7 mm. The pores may be uniform or irregular shapes and sizes.

Preferably the scaffold is porous and the plurality of channels are formed by interconnected elongated pores in the porous structure.

Preferably each or at least some of the plurality of channels extends between the first end and the second end of the scaffold.

Preferably each or at least some of the plurality of channels extend part of the way between the first end and the second end of the scaffold.

Preferably the implant is configured for implantation at a segmental defect site in a subject's long bone, the plurality of channels being configured to align substantially parallel with the longitudinal axis of said long bone when implanted.

According to a further aspect of the invention there is provided an implant for repairing a segmental defect in a subject's bone at a segmental defect site, the implant comprising: a scaffold for implantation at the segmental defect site, the scaffold comprising a first end, a second end and a longitudinal axis between the first and second ends, the scaffold being configured such that it has a first Young's modulus value in a direction of its longitudinal axis and a second Young's modulus value in a direction transverse to the longitudinal axis, the first and second Young's modulus values being different. The implant may further comprise any of the features described above or below.

The different Young's modulus value in the different directions may be due to the particular arrangement of pores in the scaffold. The scaffold is preferably anisotropic. Anisotropy can be provided to the scaffold modulus by varying pore size, shape and/or density in different directions of the scaffold.

Preferably the scaffold comprises a first end, a second end and a longitudinal axis between the first and second ends, the scaffold being configured such that it has a first Young's modulus value in a direction of its longitudinal axis and a second Young's modulus value in a direction transverse to the longitudinal axis, the first and second Young's modulus values being different.

Preferably the first Young's modulus value is greater than the second Young's modulus value.

According to a further aspect of the invention there is provided a membrane for surrounding an implant for repairing a segmental defect in a subject's bone at a segmental defect site, the implant comprising a scaffold for implantation at the segmental defect site, the membrane comprising: collagen and a resorbable polymer selected from: poly lactic acid (PLA); poly glycolic acid (PGA); poly lactic-co-glycolide (PLGA); polycaprolactone (PCL); and chitosan.

The membrane is suitably a resorbable membrane. Preferably the membrane comprises collagen and poly lactic acid (PLA). The membrane may be seeded with cells prior to implantation. Both sides of the membrane may be seeded with cells. A first type of cell may be seeded on one side and a second type of cell on the other side of the membrane.

Preferably the membrane has a first side for facing the scaffold when assembled and a second side for facing away from the scaffold when assembled, wherein the first side of the membrane is seeded with cells, preferably bone-forming cells selected from the group consisting of osteocytes, osteoblasts, osteoblast progenitor cells and stem cells.

Preferably the membrane has a first side for facing the scaffold when assembled and a second side for facing away from the scaffold when assembled, wherein the second side of the membrane is seeded with cells. The second side of the membrane may be seeded with cells such as fibroblasts or endothelial progenitor cells (EPCs).

Preferably the membrane incorporates antibiotics and/or bactericidal compounds.

According to a further aspect of the invention there is provided an implant according to any previous aspect of the invention described above, the implant further comprising a resorbable membrane, received around the scaffold of the implant. The membrane may be as described above.

In the various embodiments, the scaffold is preferably porous. The scaffold is preferably made of titanium mesh. The implants according to the present invention may be configured for implantation at a segmental defect site in a subject's long bone.

According to a further aspect of the invention there is provided a kit comprising two or more implants according to any aspect of the invention as described above, wherein each implant in the kit has at least one dimension that differs from the or each other implant in the kit. The installer of the implant can select the implant having the dimension or dimensions that suit the defect site at which the implant is to be installed. The implants in the kit may have differing lengths so that an implant can be selected having a length that corresponds substantially to the length of the defect site. Alternatively or additionally, the implants in the kit may have differing scaffold body diameters and/or cavity diameters so that an implant can be selected having a scaffold diameter that corresponds substantially to the diameter of the long bone and/or a cavity diameter that corresponds substantially to the medullary cavity diameter of the long bone to which the implant is to be implanted.

According to a further aspect of the invention there is provided a method of implanting an implant for repairing a segmental defect in a subject's bone at a segmental defect site, the method comprising providing an implant according to any aspect of the invention defined above and implanting it in the segmental defect site.

According to a further aspect of the invention there is provided a method of manufacturing an implant according to any aspect of the invention described above for repairing a segmental defect in a subject's bone at a segmental defect site.

### DESCRIPTION OF THE DRAWINGS

A preferred embodiment of the present invention will now be more particularly described by way of example only with reference to the accompanying drawings, wherein:

FIG. 1A is a diagrammatic view of an implant according to an embodiment of the invention, the implant shown implanted in a long bone, the implant including an internal fixation plate attached to a scaffold;

FIG. 1B is a diagrammatic view of an implant according to a further embodiment, the implant shown implanted in a long bone, the implant including an internal fixation plate integral with the scaffold;

FIG. 2 is a diagrammatic view of a scaffold according to an embodiment the invention;

FIG. 3 is a diagrammatic close-up view of part of a membrane and scaffold according to an embodiment of the invention;

FIG. 4 is a perspective view of a further embodiment of an implant according to the invention, the implant shown implanted in a long bone;

FIG. 5 is a perspective view of the implant of FIG. 4.

### DESCRIPTION OF THE PREFERRED EMBODIMENTS

The present embodiments represent currently the best ways known to the applicant of putting the invention into practice. But they are not the only ways in which this can be achieved. They are illustrated, and they will now be described, by way of example only.

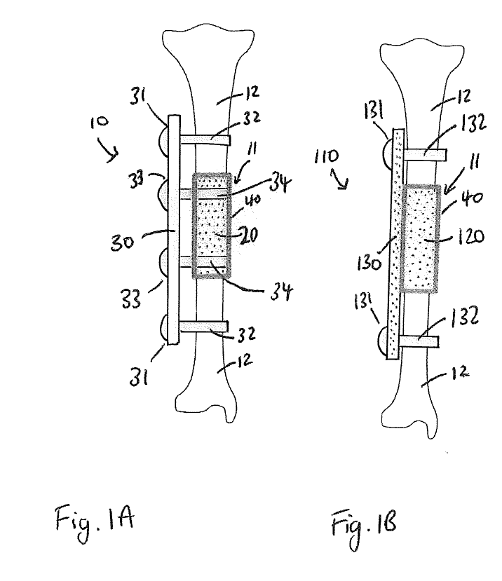

Referring to FIGS. 1A and 1B, two schematic diagrams of implants according to the invention for repairing a segmental defect in a subject's bone at a segmental defect site are shown. In each of the embodiments shown in FIGS. 1A and 1B, the implant 10, 110 comprises a scaffold 20, 120 for implantation into bone at a segmental defect site 11 and a plate 30, 130 for internal fixation. In the embodiment of FIG. 1A, the plate 30 is attachable to the scaffold 20 and to the bone 12 outside of the segmental defect site 11. In the embodiment of FIG. 1B, the plate 130 is integral with the scaffold 120. In the embodiments shown in FIGS. 1A and 1B, the bone 12 is a long bone and the scaffold 20, 120 is a spacer to be received in a bone defect between first and second long bone elements, but implants according to the invention could be employed to repair defects in other types of bone other than long bones.

In the embodiments of FIGS. 1A and 1B the scaffold 20, 120 is a selective laser sintered 3D titanium mesh but other types of scaffold can be used as will be described later. The scaffold 20, 120 stabilises the segmental defect 11 and the plate 30, 130 attaches to the subject's bone surface proximally and distally to the defect 11. The plate 30, 130 stabilises the implant via internal fixation (i.e. the plate 30, 130 is implanted internally rather than external of the body).

Referring to FIG. 1A, in this embodiment the plate has through holes 31 for receiving screws 32 to attach the plate to bone and through holes 33 for receiving screws 34 to attach the plate to the scaffold 20. Screws 32 and 34 may be the same type of screws, or different screws for attachment to bone and to the scaffold may be employed. The plate 30 may be of the type known as a “LISS” plate (Less Invasive Stabilization System) wherein at least the through holes 31 for receiving screws for attachment to bone, and optionally also the through holes 33, are internally threaded for receiving locking screws that have externally threaded heads. The plate 30 is preferably secured such that it is spaced away from the surface of the bone 12 such that it does not contact the bone 12 when implanted. The through holes 33 for receiving screws 34 for attaching the plate 30 to the scaffold 20 are in a scaffold portion 35 of the plate which overlies the scaffold 20 when implanted. The screws 34 for attaching the plate to the scaffold may be locking screws or other types of screws. FIGS. 1A and 1B are diagrammatic and the implant may have more or fewer screw holes than shown in the diagram.

The scaffold 20 preferably includes a solid or less porous region at the or each location where a screw 34 is to be received by the scaffold 20, the solid or less porous region including an internally threaded bore to receive the screw 34. This provides a stable site for the screw 34 to secure to.

Alternatively, instead of attaching the plate 30 to the scaffold 20 using screws, some other attachment means may be used. For example, the plate 30 may be attached to the scaffold 20 using at least one tape or strap that is received around plate 30 and scaffold 20 to hold them together.

The plates 30, 130 may be shaped and sized to suit the geometry of the bone they are to be attached to. For example a plate for attachment to a long bone will be elongate, having a proximal extension portion to extend proximal of the scaffold and a distal extension portion to extend distal of the scaffold, the proximal extension and distal extension portions of the plate being attachable to the bone. The plate may be bent or otherwise shaped to suit the shape of the underlying bone.

Referring to FIG. 1B, in this embodiment the plate 130 and scaffold 120 are integrally formed. The plate 130 therefore has no through holes for receiving screws for attachment of the plate to the scaffold 120. Instead, the scaffold 120 and plate 130 are portions of a one-piece, unitary construction wherein the scaffold portion 120 is monolithic with the plate portion 130. The plate 130 has through holes 131 for receiving screws 132 to attach the plate to bone. Like the implant 10 of FIG. 1A, the screws 131 may be locking screws having externally threaded heads for engagement with internally threaded through holes in the plate 130. Preferably the plate 130 closely faces the bone or contacts the bone outside of the segmental defect site 11 when implanted.

The scaffold 20, 120 will now be further described. The scaffold 20, 120 may be a porous metallic scaffold or it may be a polymeric scaffold, such as a scaffold made of a resorbable polymer. In preferred embodiments the scaffold is a selective laser sintered 3D titanium mesh. The scaffold may be a hybrid scaffold produced using both titanium and other materials such polymers. The mesh scaffold is preferably coated with hydroxyapatite. Coating with hydroxyapatite may be via an electrochemical process. Such a coating promotes osteoconduction after the scaffold has been implanted. The scaffold is preferably shaped and sized to be received at the segmental defect site 11 and to engage the native bone around the defect site. The scaffold can be custom made to suit the particular subject's defect site. If the implant includes a plate for internal fixation, the plate may be custom made for the particular subject or may be a standard piece. For an implant for repairing a bone defect in a long bone, the scaffold may preferably be substantially cylindrical in shape.

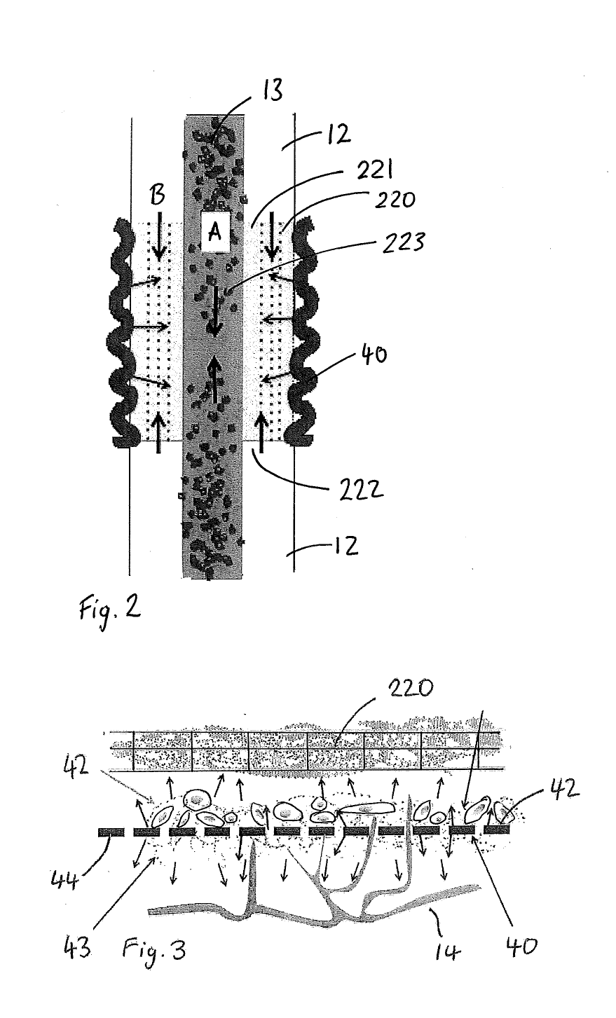

The Scaffold may optionally have a hollow void within its body surrounded by a porous or non-porous body of the scaffold. FIG. 2 shows a diagrammatic representation of a scaffold 220 that may be used with the plates shown in FIGS. 1A and 1B or in implant embodiments without internal fixation plates. For scaffolds 220 that have an internal void, preferably the scaffold 220 has first and second open ends 221, 222 and a hollow cavity 223 extending between. The scaffold 220 is sized such that first and second open ends 221 and 222 engage the bone 12 distally and proximally to the defect site. The cavity 223 may be substantially empty when it is implanted or alternatively allograft/autograft material (not shown in the diagram of FIG. 2) can be packed into the hollow cavity 223 in the scaffold 220 before it is implanted in the subject. A scaffold 220 with an engineered cavity 223 like that shown in FIG. 2 allows the invasion of bone marrow 13 into the graft from the subject's bone 12 (represented by arrows A in FIG. 2). The cavity 223 also aids bone ingrowth into the scaffold 220 from the inside of the cavity.

The scaffold 220 has a longitudinal axis running between first and second ends 221, 222 (whether these be open ends, as in a scaffold with a hollow cavity as shown in FIG. 2 or a non-hollow scaffold). The scaffold 220 may have a plurality of channels or tubes within its body, the plurality of channels being substantially aligned with the longitudinal axis of the scaffold. The channels are indicated diagrammatically in FIG. 2 by arrows B. The channels are microtubes, preferably each having a diameter of up to around 1000 micrometres, preferably around 150 to 400 micrometres, more preferably around 200 to 350 micrometres. The channels may be formed by interconnecting pores in a porous scaffold. For example the pores forming the channels may be elongated along the longitudinal axis of the scaffold. Each or some of the channels may extend fully between the first and second ends 221, 222 of the scaffold or only part way between the first and second ends 221, 222.

The scaffold 220 may be made of oriented mesh such that it directs bone in a longitudinal fashion. This is particularly useful in implants for repair of bone defects in long bones. An oriented mesh allows for the mesh to have a different Young's modulus in different directions depending on the mesh orientation. The scaffold is therefore anisotropic. The overall Young's modulus of the scaffold could be engineered to enhance osteocoductivity by controlling the strut thickness in the porous scaffold structure, thereby reducing the modulus of the implant appropriately for bone formation. For example, the scaffold may have a first Young's modulus value in a direction of its longitudinal axis and a second Young's modulus value in a direction transverse to the longitudinal axis, the first Young's modulus value preferably being greater than the second Young's modulus value.

Referring to FIGS. 1A and 1B, implants according to any of the embodiments described herein may optionally have a membrane 40 received around the scaffold before it is implanted. The membrane comprises collagen in combination with a biodegradable polymer selected from poly lactic acid (PLA); poly glycolic acid (PGA); poly lactic-co-glycolide (PLGA); polycaprolactone (PCL); and chitosan. Preferably the biodegradable polymer is provided as a porous mesh sheet onto which collagen coated. The collagen may be coated on both sides of the porous mesh sheet or on one side. The collagen fibres may intersperse through the porous mesh sheet. The membrane is preferably resorbable. The membrane is capable of being sutured. Preferably the membrane comprises collagen and poly lactic acid (PLA).

Antibiotics may be incorporated into the membrane, for which the collagen would act as a carrier, whereby the antibiotics may leach from the collagen as it is resorbed. The collagen may be cross-linked to control the rate of resorption. Additionally or alternatively the porous mesh sheet, such as a PLA sheet, can be made by 3D printing, by casting a thin film or electro spinning. The porous mesh sheet may contain a bactericidal agent such as silver or an antibiotic. The membrane is preferably porous, allowing blood vessel ingrowth to the scaffold.

The membrane 40 has a first side for facing the scaffold when assembled and a second side for facing away from the scaffold when assembled. One or both sides of the membrane 40 may be preferably seeded with cells. One or both sides may be seeded with bone forming cells such as stem cells or stem cells that have been differentiated into osteoblasts progenitor cells prior to seeding. Preferably only one side of the membrane is seeded with bone forming cells. Preferably the first side, facing the scaffold is seeded with bone forming cells prior to placement of the scaffold within the membrane. FIG. 3 shows diagrammatically a cross-section through part of a porous scaffold 220 with membrane. Stem cells or osteoprogenitor cells 42 are shown seeded on the first side 41 of the membrane. Blood vessels 14 are shown passing through the resorbable porous membrane 40. The membrane 40 is shown as comprising a porous mesh sheet 44 onto both sides of which is incorporated collagen fibres 43, which intersperse through the pores in the porous mesh sheet 44.

In addition to the seeding of bone forming cells shown in FIG. 3 onto the first side of the membrane 40, there may be cells seeded onto the second side of the membrane 40, which may also be bone forming cells or may be other cells such as fibroblasts or endothelial progenitor cells (EPCs).

In operation, in order to make and implant an implant according to the invention, firstly a scaffold is custom manufactured or selected from a standard set of pre-manufactured scaffolds. The scaffold may be packed with allograft or autograft. A membrane is formed as described above and optionally seeded with cells as described above. The membrane is wrapped around the scaffold. The membrane may be secured to the scaffold in some way, for example using sutures or using a tie or strap that wraps around outside of the membrane to hold it around the scaffold. The sutures/tie or strap may be resorbable. The scaffold is implanted into the defect site. A plate may optionally be installed for internal fixation as described above.

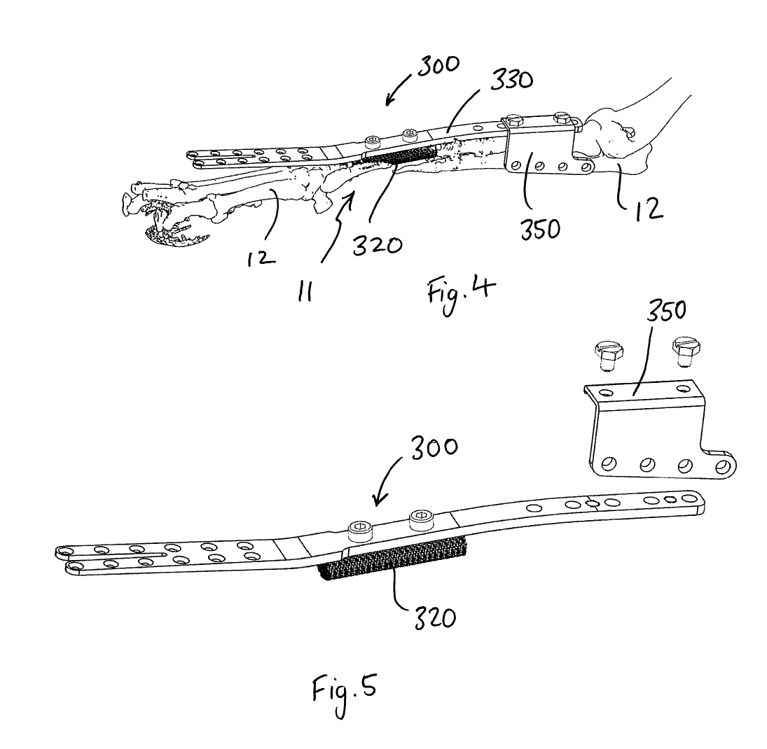

Referring to FIGS. 4 and 5, a further embodiment of an implant 310 according to the invention is shown. The implant has a titanium mesh scaffold 320 for implantation between proximal and distal parts of a long bone 12 with a bone defect 11 therebetween. The implant 310 has a plate 330 for attachment to the scaffold 320 and to the bone 12 using screws. The plate 330 is shaped to suit the geometry of the bone around the defect site 11. One end of the plate has an additional plate 350 affixed to it for fixing to the bone via screws for extra fixation.

It will be understood that various modifications, additions and alterations may be made to the invention by one skilled in the art without departing from the spirit and scope of the invention as defined in the appended claims.

1. An implant for repairing a segmental defect in a subject's bone at a segmental defect site, the implant comprising:
a scaffold for implantation at the segmental defect site, the scaffold comprising a body, the body having a first end and a second end, the first and second ends being first and second open ends respectively, the scaffold further comprising a cavity extending between said first and second open ends.

2. An implant according to claim 1, wherein said first and second ends of the scaffold are opposing ends.

3. An implant according to claim 1, wherein each of said first and second open ends of the scaffold is configured to engage bone at the segmental defect site when implanted.

4. An implant according to claim 1, wherein each of said first and second open ends of the scaffold is configured to abut bone at the segmental defect site when implanted.

5. An implant according to claim 1, wherein the length of the scaffold between its first and second ends is selected such that the first end of the scaffold abuts bone at one side of the segmental defect site and the second end of the scaffold abuts bone at the other side of the segmental defect site when implanted.

6. An implant according to claim 1, wherein the implant is configured for implantation at a segmental defect site in a subject's long bone.

7. An implant according to claim 6, wherein the cavity in the scaffold has a longitudinal axis, the implant being configured such that the longitudinal axis of the cavity substantially aligns with a longitudinal axis of the subject's long bone at the defect site when implanted in said long bone.

8. An implant according to claim 6, wherein the cavity is configured to align with the medullary cavity of said long bone when implanted.

9. An implant according to claim 6, wherein the cross-sectional size of the cavity is selected to substantially correspond with the cross-sectional size of the medullary cavity of said long bone when implanted.

10. An implant according to claim 6, wherein the cross-sectional size of the scaffold is selected to substantially correspond with the cross-sectional size of said long bone.

11.-17. (canceled)

18. An implant according to claim 1, wherein the scaffold further comprises a plurality of channels within its body, the plurality of channels being substantially aligned with a longitudinal axis of the scaffold.

19. (canceled)

20. An implant according to claim 18, wherein the scaffold is porous and the plurality of channels are formed by interconnected pores in the porous structure.

21.-25. (canceled)

26. An implant according to claim 1, wherein the scaffold is configured such that it has a first Young's modulus value in a direction of its longitudinal axis and a second Young's modulus value in a direction transverse to the longitudinal axis, the first and second Young's modulus values being different.

27. (canceled)

28. An implant according to claim 32, the membrane comprising:
collagen and
a resorbable polymer selected from: poly lactic acid (PLA); poly glycolic acid (PGA);
poly lactic-co-glycolide (PLGA); polycaprolactone (PCL); and chitosan.

29. An implant according to claim 28, wherein the membrane has a first side for facing the scaffold when assembled and a second side for facing away from the scaffold when assembled, wherein the first side of the membrane is seeded with cells, preferably bone-forming cells selected from the group consisting of osteocytes, osteoblasts, osteoblast progenitor cells and stem cells.

30. An implant according to claim 28, wherein the membrane has a first side for facing the scaffold when assembled and a second side for facing away from the scaffold when assembled, wherein the second side of the membrane is seeded with cells.

31. An implant according to claim 28, wherein the membrane incorporates antibiotics and/or bactericidal compounds.

32. An implant according to claim 1, the implant further comprising a resorbable membrane received around the scaffold of the implant.

33. (canceled)

34. An implant according to claim 1, wherein the scaffold is porous.

35. An implant according to claim 1, wherein the scaffold is made of titanium mesh.

36.-41. (canceled)
